(19) United States
(12) Patent Application Publication
(10) Pub. No.: US 2017/0182304 A1
(43) Pub. Date: Jun. 29, 2017

(54) MEDICAL PORT WITH REPLACEABLE CATHETER

(71) Applicant: Avent, Inc., Alpharetta, GA (US)

(72) Inventors: Alison S. Bagwell, Alpharetta, GA (US); Emily A. Reichart, Milford, OH (US)

(21) Appl. No.: 15/308,631

(22) PCT Filed: Apr. 29, 2015
(86) PCT No.: PCT/US2015/028194

§ 371 Date: Nov. 3, 2016

Related U.S. Application Data
(60) Provisional application No. 61/989,659, filed on May 7, 2014.

(51) Int. Cl. A61M 39/02; A61M 39/10; A61M 39/24; A61M 25/06

(52) U.S. Cl. CPC A61M 39/0247; A61M 25/0668; A61M 39/10; A61M 39/24; A61M 2039/0255; A61M 2039/0297; A61M 2039/027; A61M 2039/0273; A61M 2039/0276; A61M 2039/0261; A61M 2039/0291

(57) ABSTRACT

The present disclosure is directed to a medical port drainage assembly that includes a port component and a replaceable catheter. The port component includes a tube having a proximal end, a distal end, and tube walls defining a catheter access lumen. A head component is located on a proximal end of the tube and an assembly retention element is located on a distal end of the tube. The head component is deployed outside the patient's body and defines an opening to the catheter access lumen. The assembly retention element is deployed within a lumen of the patient's body. The port component includes a valve assembly which may be a single valve (e.g., a duckbill valve) or a system of valves. The port component also includes a connector for coupling and decoupling the replaceable catheter to the port component.

### RELATED APPLICATIONS

The present application claims priority to U.S. Provisional Application Ser. No. 61/989,659 filed on May 7, 2014, which is incorporated herein in its entirety by reference hereto.

### FIELD OF THE INVENTION

This invention relates to an apparatus or equipment for draining fluid from a body cavity of a patient and methods of using such apparatus or equipment to drain fluid from a body cavity. More specifically, the invention relates to an apparatus or used for paracentesis and methods of using such apparatus or equipment to perform paracentesis.

### BACKGROUND

Clogging is a known problem for tubes used in drainage delivery systems, bowel management, nutrition, and/or dialysis. For example, many patients that require paracentesis procedures require them on a continuous basis and an in-dwelling catheter is placed for repeated access. Currently, such catheters are placed utilizing a tunneling technique. The tunneled catheters contain a polyester cuff at the most proximal end to aid in promoting tissue growth and to act as a mechanical obstacle to bacterial infiltration. The polyester cuff is known to reduce infection of the tract; however, the cuff can make it difficult to remove the catheter. When a tunneled catheter is clogged, kinked, or if the position of the tube is not allowing for proper drainage, there is a need to replace the catheter. When the catheter is damaged, it is necessary to remove the catheter and insert a new one. Replacing the catheter can cause additional trauma to the patient, thereby increasing the risk of infection.

A common example of the tunneling procedure includes a guide wire introducer with a needle inserted therethrough that is inserted through the abdominal wall at the desired insertion site under fluoroscopic guidance. The needle is removed while the guide wire introducer is left in place. An initial incision is made through the guide wire insertion site. A second incision is made from about 5 to about 8 centimeters (cm) from the initial incision. A tunneler/catheter assembly is passed subcutaneously from the second incision down to and out through the incision at the guide wire insertion site until the polyester cuff on the catheter lies about 1 cm inside the second incision. The insertion site is dilated and a peel away introducer sheath is threaded over the guide wire and advanced into the peritoneal space. The guide wire and dilator are removed as a unit and the peel-away introducer sheath is left in place. The fenestrated end of the catheter is advanced into the sheath until all the fenestrations are within the peritoneal cavity. This can be verified under fluoroscopy as fenestrations are located along the barium sulfate stripe. The peel-away sheath is removed and the incision is closed at the insertion site. The catheter is then typically sutured to the skin superior to the second incision. Variations to the tunneling procedure can be performed, for example, retrograde, antegrade, and/or over the wire. However, all procedures utilize two incisions and the tunneling technique.

Infection is the most common complication associated with tunneled catheters as the procedure requires two incisions which increases the risk of infection at the exit site. In addition, the distal end of the catheter is placed into the peritoneal cavity which can lead to peritonitis, a serious condition that can lead to patient death. Accordingly, the present invention is directed to a medical port drainage assembly for drainage of fluids that addresses the aforementioned issues.

### SUMMARY

The present disclosure is directed to a medical port drainage assembly that includes a port component and a replaceable catheter. The port component includes a tube having a proximal end, a distal end, and tube walls defining a catheter access lumen. In addition, the port component includes a head component configured with the proximal end of the tube and an assembly retention element configured with the distal end of the tube. As such, when the drainage assembly is inserted into a patient, the head component is outside the patient's body and defines an opening to the catheter access lumen. Further, the assembly retention element is within the patient's body, e.g. in a lumen thereof. The port component includes a valve assembly which may be a single valve (e.g., a duckbill valve) or a system of valves. Further, the port component also includes a connector for coupling and decoupling the replaceable catheter to the port component.

The replaceable catheter is configured to pass through the catheter access lumen of the port component and engage the assembly retention element (e.g. via the connector) such that the catheter is releasably coupled to the port component. In this manner, the replaceable catheter may be removed and replaced through the catheter access lumen of the port component.

In further embodiments, the assembly retention element may be a funnel-shaped cross-section that is configured to expand and assist in leakage prevention in addition to providing a retention means. Further, in certain embodiments, the assembly retention device is constructed of a foam material. Alternatively and/or additionally, the assembly retention element may be a foam-filled balloon, an inflatable balloon, a fibrous polyester ring, and combinations thereof.

In additional embodiments, the connector may be a clamp, a friction fitting, a press fitting, a snap fitting configured to engage an element on the catheter, or a screw fitting configured to engage an element on the catheter.

In another aspect, the present disclosure is directed to a method for placing a medical port drainage assembly for drainage of fluids from a patient's body. More specifically, the method includes dilating a stoma tract of the patient's body. Another step includes inserting a tubular peel-away introducer sheath into the stoma tract such that the sheath is entirely within the dilated stoma tract. The method also includes inserting the medical port drainage assembly through the peel-away sheath such that the head component of the drainage assembly is outside of the patient's body and the assembly retention element is within the patient's body, e.g. in a lumen thereof. Still another step includes removing the peel-away sheath from the stoma tract. Further, the method includes activating the assembly retention element of the medical port drainage assembly.

In one embodiment, the step of dilating the stoma tract of the patient's body may include utilizing a serial dilator. In another embodiment, the step of activating the assembly retention element of the medical port drainage assembly may further include inserting a replaceable catheter within a lumen of the port component and engaging the replaceable catheter with the assembly retention element such that the replaceable catheter is releasably coupled to the port component.

In still another embodiment, the assembly retention element may include a connector. Thus, in certain embodiments, the method may include engaging the replaceable catheter with the connector of the assembly retention element such that the replaceable catheter is releasably coupled to the port component.

In certain embodiments, the tubular peel-away sheath may include a split valve for restricting fluid flow. Further, in particular embodiments, the split valve may include a breakaway valve body.

Another embodiment of present disclosure is directed to a method for placing a medical port drainage assembly for drainage of fluids from a patient's body. The method includes dilating, via a serial dilator, a stoma tract of the patient. Further, the serial dilator may include a split valve. Another step includes inserting a tubular peel-away introducer sheath such that the sheath is entirely within the dilated stoma tract. The method also includes coupling the split valve with the tubular peel-away sheath so as to form an introducer sheath assembly. Still another step includes inserting the medical port drainage assembly through the introducer sheath assembly such that the head component of the drainage assembly is outside of the patient's body and the assembly retention element is within the patient's body during use. The method also includes removing the peel-away sheath from the patient's body. Another step includes activating the assembly retention element of the medical port drainage assembly.

In one embodiment, the step of activating the assembly retention element of the medical port drainage assembly may further include inserting a replaceable catheter within a lumen of the port component and engaging the replaceable catheter with the assembly retention element such that the replaceable catheter is releasably coupled to the port component. Further, in certain embodiments, the assembly retention element may include a connector. Thus, the method may also include engaging the replaceable catheter with the connector of the assembly retention element such that the replaceable catheter is releasably coupled to the port component.

Further, as mentioned, the split valve may include a breakaway valve body.

These and other features, aspects and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE FIGURES

A full and enabling disclosure of the present invention, including the best mode thereof, directed to one of ordinary skill in the art, is set forth in the specification, which makes reference to the appended figures, in which:

FIG. 1 illustrates a partial, perspective view of one embodiment of a medical port drainage assembly for drainage of fluids from a patient's body according to the present disclosure, particularly illustrating a peel-away sheath that may be used to insert the assembly within the patient's body;

FIG. 2 illustrates a partial, perspective view of the medical port drainage assembly of FIG. 1, particularly illustrating the peel-away sheath being removed from the medical port drainage assembly;

FIG. 3 illustrates a perspective view of one embodiment of the medical port drainage assembly according to the present disclosure, particularly illustrating a replaceable catheter configured with the port component of the medical port drainage assembly;

FIG. 4 illustrates a detailed, perspective view of the medical port drainage assembly of FIG. 1, particularly illustrating the head component of the assembly;

FIG. 5 illustrates a flow diagram of one embodiment of a method for placing a medical port drainage assembly for drainage of fluids from a patient's body according to the present disclosure; and

FIG. 6 illustrates a flow diagram of another embodiment of a method for placing a medical port drainage assembly for drainage of fluids from a patient's body according to the present disclosure.

### DETAILED DESCRIPTION

Reference will now be made in detail to one or more embodiments, examples of which are illustrated in the drawings. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used with another embodiment to yield a still further embodiment. Thus, it is intended that the present invention covers such modifications and variations as come within the scope of the appended claims and their equivalents.

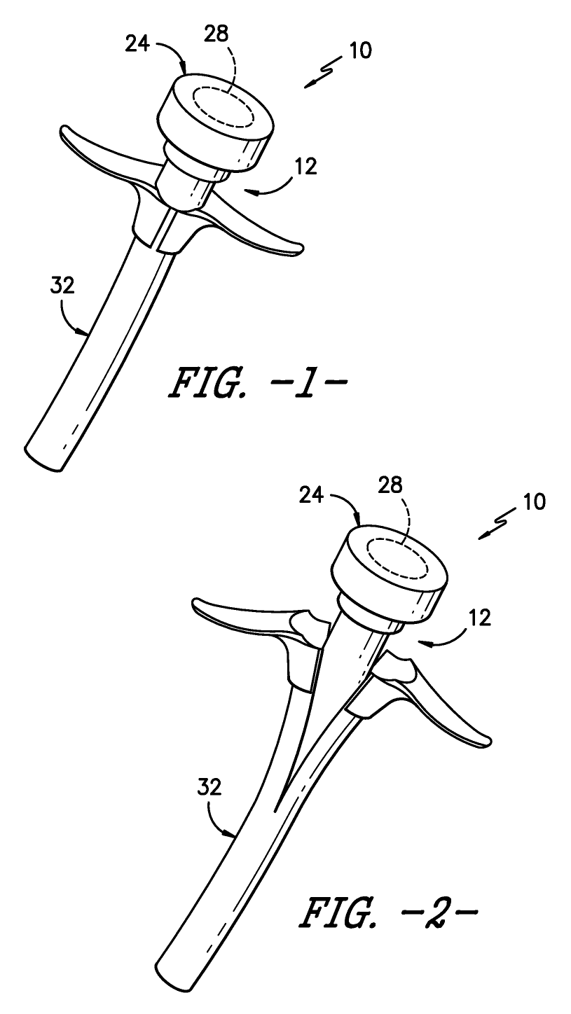

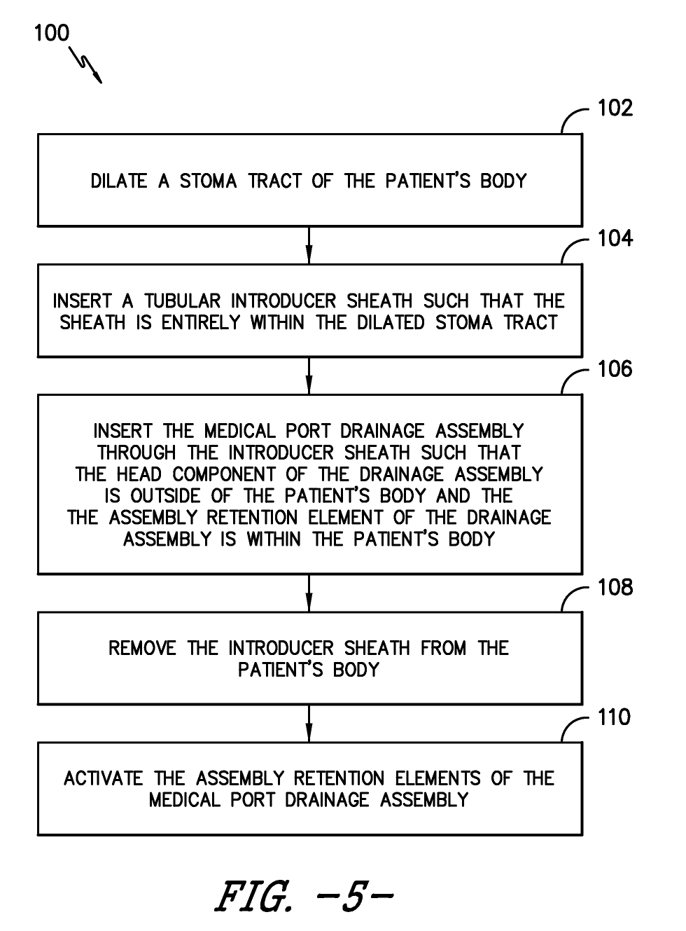

Referring now to the drawings, FIGS. 1-4 illustrate various embodiments of a medical port drainage assembly 10 according to the present disclosure. While the medical port drainage assembly 10 is desirably configured for drainage of the abdominal space such as for paracentesis procedures, the assembly 10 may be placed for bowel management, nutrition, dialysis, and/or drainage delivery systems. As shown, the medical port drainage assembly 10 generally includes a port component 12 and a replaceable catheter 14 (e.g., a drainage catheter or the like which may become damaged and/or clogged during use) as particularly shown in FIG. 3. The port component 12 includes a tube 16 having a proximal end 18, a distal end 20, and tube walls defining a catheter access lumen 22. Further, the drainage assembly 10 includes a head component 24 configured with the proximal end 18 of the tube 16 and an assembly retention element 26 configured with the distal end 20 of the tube 16. During use, the head component 24 is outside of the patient's body and defines an opening 28 to the catheter access lumen 22. Thus, the replaceable catheter 14 can be easily inserted and removed through the catheter access lumen 22 when the port component 12 of the drainage assembly 10 is located within a patient. Further, the assembly retention element 26 remains within the patient's body, e.g. in a stoma tract of the patient, during use, and is configured to retain the replaceable catheter 14 therein.

In addition, the port component 12 includes a valve assembly which may be a single valve (e.g., a duckbill valve) or a system of valves. Further, the assembly retention element 26 of the port component 12 may also include a connector 30 for coupling and decoupling the replaceable catheter 14 to the port component 12. As such, the present disclosure allows a catheter to be easily coupled to the port component 12 and replaced upon damage.

More specifically, the replaceable catheter 14 is configured to pass through the catheter access lumen 22 of the port component 12 and engage the connector 30 to be releasably coupled to the port component 12. In this manner, the replaceable catheter 14 may be removed and replaced through the catheter access lumen 22 of the port component 12.

Referring particularly to FIG. 3, the assembly retention element 26 as described herein may have a funnel-shaped cross-section that is configured to expand and assist in leakage prevention in addition to providing a retention means. More specifically, in certain embodiments, the funnel-shaped assembly retention element 26 may include a foam material to assist in further leakage protection. Alternatively and/or additionally, the assembly retention element 26 may be a foam-filled balloon, an inflatable balloon, a fibrous polyester ring, and combinations thereof. For example, the fibrous polyester ring will aid in the promotion of tissue in-growth to prevent the spread of infection.

In certain embodiments, as mentioned, the connector 30 as described herein may include a clamp, a friction fitting, a press fitting, a snap fitting configured to engage an element on the catheter, a screw fitting configured to engage an element on the catheter, or similar.

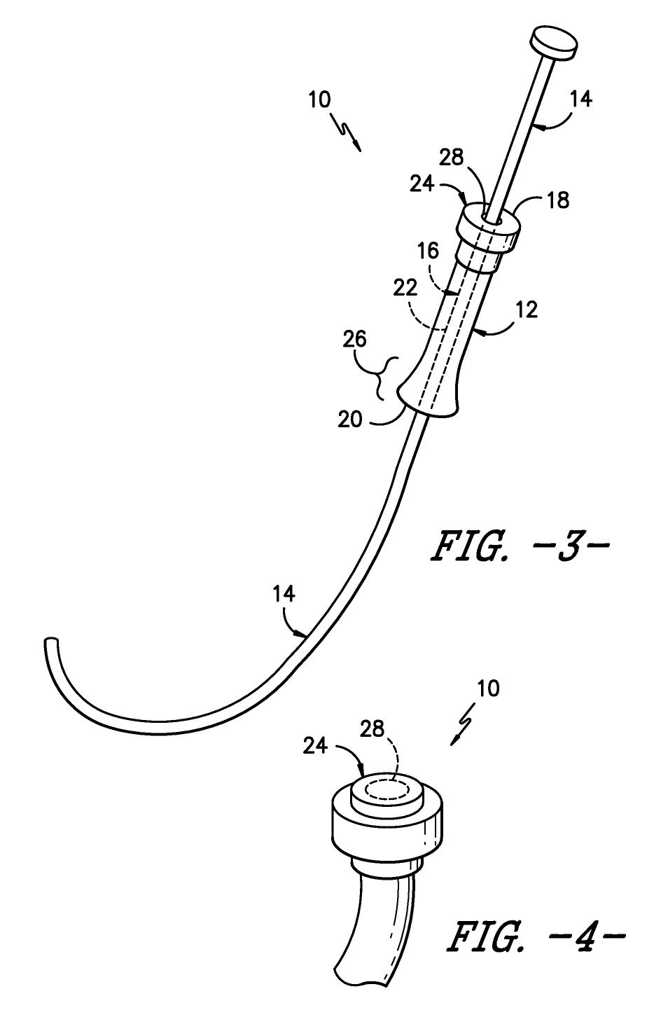

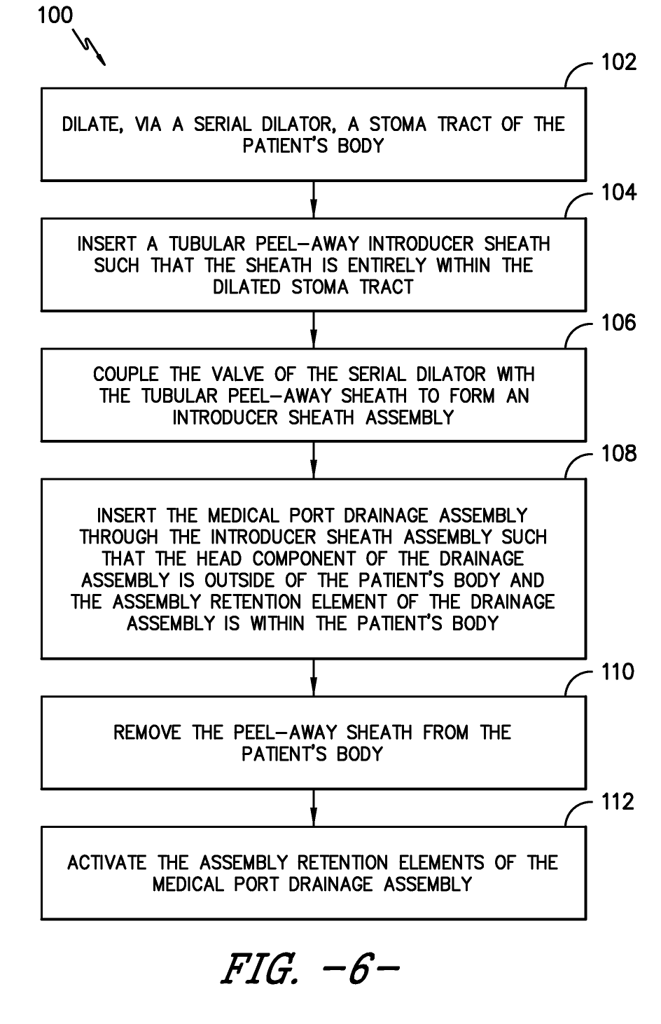

Referring now to FIG. 5, a flow diagram of a method 100 for placing a medical port drainage assembly for drainage of fluids from a patient's body according to the present disclosure is illustrated. In general terms and with respect to paracentesis procedures, the medical port drainage assembly 10 is placed percutaneously into the peritoneal space via a peel-away sheath (as shown in FIGS. 1 and 2) and/or via direct placement, e.g. utilizing the Seldinger technique. More specifically, as shown at 102, the method 100 includes dilating a stoma tract of the patient's body. At 104, the method 100 also includes inserting a tubular introducer sheath 32 (e.g. a peel-away sheath) such that the sheath 32 is entirely within the dilated stoma tract. At 106, the method 100 includes inserting the medical port drainage assembly 10 of the present disclosure through the introducer sheath 32 such that the head component 24 of the drainage assembly 10 is outside of the patient's body and the assembly retention element 26 of the drainage assembly 10 is within the patient's body, for example, as shown in FIG. 1. At 108, the method 100 includes removing the introducer sheath 32 from the patient's body, for example, as shown in FIG. 2. At 110, the method 100 includes activating the assembly retention element 26 of the medical port drainage assembly 10, e.g. by inserting a replaceable catheter 14 through the port component 12 and engaging the catheter 14 with the assembly retention element 26 thereof.

In certain embodiments, the tubular introducer sheath 32 may include a split valve for restricting fluid flow. In additional embodiments, the split valve may desirably include a breakaway valve body. Exemplary tissue dilation systems may be found at, for example, PCT International Application Publication WO 2009/027859 A1 entitled “Stoma Dilator” by Griffith et al., published Mar. 5, 2009; PCT International Application Publication WO 2007/125488 A1 entitled “Percutaneous Dilation Apparatus” by McMichael et al., published Nov. 8, 2007; and PCT International Application Publication WO 2007/125440 A1 also entitled “Percutaneous Dilation Apparatus” by McMichael et al., published Nov. 8, 2007; the entire contents of each are incorporated herein by reference. Exemplary split valves for peel away sheaths are described at, for example, U.S. Pat. No. 7,303,552 for “Split Valve for Peel-Away Sheath” issued Dec. 4, 2007 to Chu et al., the entire contents of which is incorporated herein by reference.

Referring now to FIG. 6, a flow diagram of another embodiment of a method 200 for placing a medical port drainage assembly 10 for drainage of fluids from a patient's body is illustrated. As shown at 202, the method 200 includes dilating, via a serial dilator, a stoma tract of the patient's body. More specifically, in certain embodiments, the serial dilator may incorporate a split valve. At 204, the method 200 includes inserting a tubular introducer sheath 32 (e.g. a peel-away sheath) such that the sheath is entirely within the dilated stoma tract. At 206, the method 200 includes coupling the split valve of the serial dilator with the tubular introducer sheath to form an introducer sheath assembly. At 208, the method 200 includes inserting the medical port drainage assembly 10 through the introducer sheath assembly such that the head component of the drainage assembly is outside of the patient's body and the assembly retention element of the drainage assembly is within the patient's body. At 210, the method 200 includes removing the introducer sheath from the patient's body. At 212, the method 200 includes activating the assembly retention element of the medical port drainage assembly 10, e.g. by inserting a replaceable catheter 14 through the port component 12 and engaging the catheter 14 with the assembly retention element 26 thereof. In addition, as mentioned, the split valve as described herein may include a breakaway valve body.

The serial dilation system provides two or more tissue dilation tubes telescopically arranged and movable relative to each other. Additionally, the split valve peel-away sheaths help prevent backflow and/or spray back when used in drainage and/or vascular procedures. One advantage of the serial dilation system is the reduction in the number of steps required to perform the procedure. The present invention incorporates such technologies into a system used in drainage tube placement procedures. The split valve technology is incorporated into the end of the serial dilation system. The inner assembly is combined through the valve prior to placement to ensure that the structural integrity of the seal remains intact prior to use of the system. In particular, the method of the present disclosure is particularly advantageous for placing the port component of the drainage assembly that can be used for paracentesis drainage procedures. This is helpful for this procedure due to the tract dilation requirements necessary to place the port component.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

1. A medical port drainage assembly for drainage of fluids from a patient's body, the drainage assembly comprising:
a port component comprising:
a tube having a proximal end, a distal end, and tube walls defining a catheter access lumen,
a head component configured with the proximal end of the tube and defining an opening to the catheter access lumen, the head component being configured outside of the patient's body during use, and
an assembly retention element located on a distal end of the tube, the assembly retention element being configured within the patient's body during use; and
a replaceable catheter configured to pass through the catheter access lumen of the port component and engage the assembly retention element such that the replaceable catheter is releasably coupled to the port component,
wherein the replaceable catheter is removed and replaced through the catheter access lumen of the port component.

2. The drainage assembly of claim 1, wherein the port component further comprises a connector configured with the assembly retention element, wherein the replaceable catheter engages the connector.

3. The drainage assembly of claim 1, further comprising a valve assembly.

4. The drainage assembly of claim 3, wherein the valve assembly comprises a duckbill valve.

5. The drainage assembly of claim 3, wherein the valve assembly comprises a system of valves.

6. The drainage assembly of claim 1, wherein the assembly retention element comprises a funnel-shaped cross-section configured to expand and assist in leakage prevention in addition to providing a retention means.

7. The drainage assembly of claim 1, wherein the assembly retention element is constructed of a foam material.

8. The drainage assembly of claim 1, wherein the assembly retention element comprises at least one of a foam-filled balloon, an inflatable balloon, a fibrous polyester ring, or combinations thereof.

9. The drainage assembly of claim 2, wherein the connector comprises at least one of a clamp, a friction fitting, a press fitting, a snap fitting, or a screw fitting.

10. A method for placing a medical port drainage assembly for drainage of fluids from a patient's body, the medical port drainage assembly comprising a port component having a head component and an assembly retention element, the method comprising:
dilating a stoma tract of the patient's body;
inserting a tubular introducer sheath into the stoma tract such that the sheath is entirely within the dilated stoma tract;
inserting the medical port drainage assembly into the introducer sheath such that the head component of is outside of the patient's body and the assembly retention element is with the patient's body;
removing the introducer sheath from the patient's body; and
activating the assembly retention element of the medical port drainage assembly.

11. The method of claim 10, wherein dilating the stoma tract of the patient's body comprises utilizing a serial dilator.

12. The method of claim 10, wherein activating the assembly retention element of the medical port drainage assembly further comprises inserting a replaceable catheter within a lumen of the port component and engaging the replaceable catheter with the assembly retention element such that the replaceable catheter is releasably coupled to the port component.

13. The method of claim 10, wherein the assembly retention element further comprises a connector.

14. The method of claim 13, wherein inserting the replaceable catheter within the lumen of the port component further comprises engaging the replaceable catheter with the connector of the assembly retention element such that the replaceable catheter is releasably coupled to the port component.

15. The method of claim 10, wherein the introducer sheath comprises a split valve for restricting fluid flow.

16. The method of claim 15, wherein the split valve comprises a breakaway valve body.

17. A method for placing a medical port drainage assembly for drainage of fluids from a patient's body, the method comprising:
dilating a stoma tract of the patient's body, the serial dilator comprising a split valve;
inserting an introducer sheath such that the sheath is entirely within the dilated stoma tract;
coupling the split valve with the introducer sheath to form an introducer sheath assembly;
inserting the medical port drainage assembly through the introducer sheath assembly such that the head component of the drainage assembly is outside of the patient's body and the assembly retention element of the drainage assembly is within the patient's body;
removing the introducer sheath; and
activating the assembly retention element of the medical port drainage assembly.

18. The method of claim 17, wherein activating the assembly retention element of the medical port drainage assembly further comprises inserting a replaceable catheter within a lumen of the port component and engaging the replaceable catheter with the assembly retention element such that the replaceable catheter is releasably coupled to the port component.

19. The method of claim 17, wherein the assembly retention element further comprises a connector, and wherein inserting the replaceable catheter within the lumen of the port component further comprises engaging the replaceable catheter with the connector of the assembly retention element such that the replaceable catheter is releasably coupled to the port component.

20. The method of claim 17, wherein the split valve comprises a breakaway valve body.
